(19) United States
(12) Patent Application Publication
(10) Pub. No.: US 2022/0193181 A1
(43) Pub. Date: Jun. 23, 2022

(54) S-NITROSOGLUTATHIONE (GSNO) AND GSNO REDUCATASE INHIBITORS FOR USE IN THERAPY

(71) Applicant: MUSC FOUNDATION FOR RESEARCH DEVELOPMENT, Charleston, SC (US)

(72) Inventors: Inderjit Singh, Mount Pleasant, SC (US); Avtar K. Singh, Mount Pleasant, SC (US)

(73) Assignee: MUSC FOUNDATION FOR RESEARCH DEVELOPMENT, Charleston, SC (US)

(21) Appl. No.: 17/557,628

(22) Filed: Dec. 21, 2021

Related U.S. Application Data
(60) Provisional application No. 63/128,585, filed on Dec. 21, 2020.

(51) Int. Cl. A61K 38/06; A61K 31/198; A61K 31/4178; A61K 38/21; A61K 38/28; A61K 45/06

(52) U.S. Cl. CPC A61K 38/063; A61K 31/198; A61K 45/06; A61K 38/215; A61K 38/28; A61K 31/4178

(57) ABSTRACT

The present disclosure provides methods for the treatment or prevention of SARS-Cov-2 infection or disease and IL-6 mediated immune/autoimmune and cancers by the administration of GSNO or GSNO reductase inhibitor.

### PRIORITY CLAIM

This application claims benefit of priority to U.S. Provisional Application Ser. No. 63/128,585, filed Dec. 21, 2020, the entire content of which is hereby incorporated by reference.

### STATEMENT REGARDING FEDERALLY FUNDED RESEARCH

The invention was made with government support under Grant No. NS-22576 and NS-37766 awarded by the National Institutes of Health and Grant No. VA RR 3401 awarded by the U.S. Department of Veterans Affairs. The government has certain rights in the invention.

### BACKGROUND

### 1. Field

The present disclosure relates generally to the field of medicine. More particularly, it concerns methods of treating viral diseases by administering S-nitrosoglutathione (GSNO) and/or a GSNO reductase inhibitor.

### 2. Description of Related Art

The ongoing pandemic outbreak of SARS-Cov-2, a novel and highly contagious and lethal Corona family virus, infection has spread at an alarming rate worldwide since December 2019 leading the World health organization to declare it a global health emergency. Coronaviruses (CoVs) primarily target the human respiratory system and are known to cause mild respiratory disease as observed in the common cold; however, some other members of the CoVs family such as SARS-CoV-1 and MERS-CoV, and now SARS-Cov-2, cause severe pneumonia with lethal consequences and thus are a great Public health threat (pandemic).

It has been reported that SARS-Cov-2 infection results in a multifactorial disease affecting multiple cell/organ systems and multi mechanistic disease. Therefore, for an effective therapy the potential drug needs to target SARS-Cov-2 disease mechanisms in virus infection and its replication, imbalanced immune system such as cytokine storm, ARDS, systemic hypoxia and related vascular/endothelial integrity/dysfunction and related chronic post-SARS-Cov-2 disease manifestations. Although effective vaccines are rapidly being developed around the world, the absence of effective therapy for SARS-Cov-2 patients underscores the urgent need to develop an effective treatment including vaccine and drugs.

### SUMMARY OF THE INVENTION

Embodiments of the present disclosure concern a method of treating or preventing viral infection or disease comprising administering an effective amount of S-nitrosoglutathione (GSNO) and/or a GSNO reductase inhibitor to the subject. The GSNO reductase inhibitor may be N6022, N91115, N6338, Cavonsonstat or SPL-334.1. Treating may result in reduced hypoxia, may upregulate anti-inflammatory innate and adaptive immune response, such as wherein the upregulated anti-inflammatory innate and adaptive immune response comprises IL-10 and/or IL-4, or may downregulate pro-inflammatory innate and adaptive immune response, such as wherein the downregulated anti-inflammatory innate and adaptive immune response comprises TNFα, IL-1β and/or IL-6. Treating may result in a reduction or prevention of one or more symptoms viral infection or disease, such as fever, chills, cough, shortness of breath or difficulty breathing, fatigue, muscle or body aches, headache, loss of taste or smell, sore throat, congestion or runny nose, nausea or vomiting, diarrhea, trouble breathing or low blood oxygenation, persistent pain or pressure in the chest, confusion, pneumonia (including viral induced pneumonia) and acute respiratory distress syndrome (ARDS), blood/vascular, neurological and cognition dysfunctions, post-COVID-19 disease/dysfunction or vascular and multiorgan COVID-19 disease among “long hauler” COVID patients.

The subject may have an active viral infection or may be at risk of viral infection. The subject may have tested positive for a viral infection or have tested negative for viral infection. The GSNO and/or GSNO reductase inhibitor may be administered orally, intravenously, intraperitoneally, intratracheally, intratumorally, intramuscularly, endoscopically, percutaneously, subcutaneously, regionally, or by direct injection or perfusion. The subject may be a human or a non-human animal. The viral infection or disease may be a pulmonary viral infection or disease. The viral infection or disease may be caused by ebolavirus, hantavirus, SARS-CoV-1, SARS-CoV-2/COVID-19, MERS-CoV, influenza virus, Dengue virus, and respiratory syncytial virus.

In another embodiment, there is provided a method of treating or preventing IL-6 associated disorders in a subject including cancer, a chronic inflammation disease, an autoimmune disease condition, Castleman's disease, systemic onset juvenile idiopathic arthritis, steroid sparing rheumatoid arthritis, progressive MS, systemic sclerosis, systemic lupus erythematosus, Crohn's disease, neuromyelitis optica spectrum disorders (NMOSD), cytokine release syndrome and multi organ failure comprising administering an effective amount of S-nitrosoglutathione (GSNO) and/or a GSNO reductase inhibitor to the subject. The cancer may include an IL-6 expressing cancer of breast, prostate, pancreatic, lung, head and neck, colorectal and renal cancers, lymphoma, renal cell carcinoma, a hematopoeitic tumor, or a hypoxia associated cancer.

The GSNO reductase inhibitor may be N6022, N91115, N6338, Cavonsonstat or SPL-334.1 Treating may upregulate anti-inflammatory innate and adaptive immune response, such as wherein the upregulated anti-inflammatory innate and adaptive immune response comprises IL-10 and/or IL-4, or treating may downregulate pro-inflammatory innate and adaptive immune response, such as wherein the downregulated anti-inflammatory innate and adaptive immune response comprises TNFα, IL-1β and/or IL-6. The GSNO and/or GSNO reductase inhibitor may be administered orally, intravenously, intratumorally intraperitoneally, intratracheally, intratumorally, intramuscularly, endoscopically, percutaneously, subcutaneously, regionally, or by direct injection or perfusion. The subject may be a human or a non-human animal.

Also provided is a method of a chronic inflammatory and hypoxic disease conditions associated with increased expression and activity of GSNO reductase and thus decreased GSNO/S-nitrosylation and dysregulations of inflammatory and hypoxic cellular regulations comprising administering an effective amount of S-nitrosoglutathione (GSNO) and/or a GSNO reductase inhibitor to the subject.

As used herein, “essentially free,” in terms of a specified component, is used herein to mean that none of the specified component has been purposefully formulated into a composition and/or is present only as a contaminant or in trace amounts. The total amount of the specified component resulting from any unintended contamination of a composition is therefore well below 0.05%, preferably below 0.01%. Most preferred is a composition in which no amount of the specified component can be detected with standard analytical methods.

As used herein the specification, “a” or “an” may mean one or more. As used herein in the claim(s), when used in conjunction with the word “comprising,” the words “a” or “an” may mean one or more than one.

The use of the term “or” in the claims is used to mean “and/or” unless explicitly indicated to refer to alternatives only or the alternatives are mutually exclusive, although the disclosure supports a definition that refers to only alternatives and “and/or.” As used herein “another” may mean at least a second or more.

Throughout this application, the term “about” is used to indicate that a value includes the inherent variation of error for the device, the method being employed to determine the value, or the variation that exists among the study subjects.

Other objects, features and advantages of the present invention will become apparent from the following detailed description. It should be understood, however, that the detailed description and the specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The following drawings form part of the present specification and are included to further demonstrate certain aspects of the present invention. The invention may be better understood by reference to one or more of these drawings in combination with the detailed description of specific embodiments presented herein.

FIG. 1—Effect of GSNO and GSNOR inhibitor (N6022 and N91115) on secretion of pro- or anti-inflammatory cytokine in THP-1 derived macrophage. THP-1 cells were cultured in the presence of PMA (50 ng/ml; treated for 48 hr) for differentiation into macrophages. Following the differentiation, the cells were treated with LPS (100 ng/ml) in the presence or absence of GSNO (10 μM), N6022 (1 μM), and N91115 (1 μM) and incubated for 48 hr. GSNO, N6022, and N91115 were pretreated 1 hr before LPS treatment. The levels of IL-10, IL-6, TNF-α and IFN-7 in the media were quantified by enzyme-linked immunosorbent assay (ELISA). Bars represent the means±SEM from three independent experiments. Significant differences between means of media alone and individual stimuli are indicated: **p≤0.01, ***p≤0.001 as compared to the control group. +p≤0.05, ++p≤0.01, +++p≤0.001 as compared to LPS treated group. N.S. represents not significant.

FIG. 2—GSNO and GSNOR inhibitor (N6022 and N91115) regulates expression of IL-6 and IL-10 mRNA in human macrophage. THP-1 cells were cultured in the presence of PMA (50 ng/ml; treated for 48 hr) for differentiation into macrophages. Following the differentiation, the cells were treated with LPS (100 ng/ml) in the presence or absence of GSNO (10 μM), N6022 (1 μM), and N91115 (1 μM) and incubated for 6 hr. GSNO, N6022, and N91115 were pretreated 1 hr before LPS treatment. The levels of IL-10 and IL-6 mRNA were analyzed by quantitative PCR (qPCR). Bars represent the means±SEM from three independent experiments. Significant differences between means of media alone and individual stimuli are indicated: *p≤0.05, **p≤0.01, ***p≤0.001 as compared to the control group. +p≤0.05, ++p≤0.01, +++p≤0.001 as compared to LPS treated group. N.S. represents not significant.

FIGS. 3A-D—In vitro effect of GSNO on B cell expression of IL-10 versus IL-6. Splenic B cells purified from C57BL/6 mice were stimulated with IgM-specific goat F(ab′)2 Ab (@IgM: 10 μg/ml; Invitrogen #16509285) and co-stimulated (CS) with rBAFF (100 ng/ml; R&D #2149BF) and anti-CD40 Ab (10 μg/ml; Enzo #ALX805046) with/without GSNO (50 μM) pretreatment (1 hr). After 43 hr incubation, the cells were further incubated with PMA/ionomycin/Brefeldin-A for 5 hr. (FIG. 3A) Then, the levels of IL-10 (i) and IL-6 (ii) were analyzed by ELISA. (FIG. 3B) The numbers of B cells (B220⁺) (i), IL-10⁺ B cells (B220⁺IL-10⁺) (ii), and IL-6⁺ B cells (B220⁺IL-6⁺) (iii) were analyzed by flow cytometry. (FIG. 3C) The numbers of CD1d^{hi}CD5^{hi} B cells (B220⁺) (i), IL-10⁺ (ii) and IL-6⁺ CD1d^{hi}CD5^{hi} B cells (B220⁺) were analyzed. (FIG. 3D) The numbers of CD1d^{lo}CD5^{hi} B cells (B220⁺) (i), IL-10⁺ (ii) and IL-6⁺ CD1d^{lo}CD5^{hi} B cells (B220⁺) were analyzed. Data are expressed as mean±SEM. **p≤0.01, ***p≤0.001 vs. control mice and +p≤0.05, ++p≤0.01, +**p≤0.001 vs. as indicated. The “n” represents the number of cell cultures.

FIGS. 4A-C. B cell/ASC subset-specific effect of GSNO on the expression of IL-10 versus IL-6. (FIG. 4A) C57BL/6 mice were immunized with MOG₃₅₋₅₅ for the induction of EAE. EAE mice received daily doses of saline or N6022 (1 mg/kg/day/ip) starting day 14. On day 21, lymphocytes were isolated from the CNS (i) and spleen (ii) of control, EAE, and EAE treated with N6022. Then, the numbers of total, IL-10⁺, or IL-6⁺ B cell/ASC subsets (Bla, Bib, Breg, transitional B cells (T1, T2), marginal zone (MZ) B cells, MZ precursor B cells (MZP), follicular B cells (FO), memory B cells, plasmablasts, and plasma cells) were analyzed by flow cytometry. Please see table 1 for the phenotyping of these subsets of cells. (FIG. 4B) Human blood B cells (Stemcell cat #: 70023) were stimulated with anti-IgM/IgG Ab (@IgM: 10 μg/ml; eBioscience #16-509985) and co-stimulated with rBAFF (100 ng/ml; R&D #2149BF) and anti-CD40 Ab (1 g/ml; Enzo #ALX805046) in the presence or absence of GSNO (50 μM/1 hr) pretreatment. 43 hr after the cells were further incubated with PMA/ionomycin/Brefeldin-A for 5 hr and cells were stained for total B cells, transitional/immature B cells, mature B cells, memory B cells, plasmablasts, or plasma cells and their expression of IL-10 and IL-6. The graphs show mean±SEM: * p≤0.05, ** p≤0.001, ***p≤0.0001 vs. control and +p≤0.05, ++p≤0.01, +++p≤0.001 vs. as indicated. (n.s.: not significant).

FIGS. 5A-G. Effect of N6022 and GSNO on B cell expression of IL-10 versus IL-6 in EAE. C57BL/6 mice were immunized with MOG₃₅₋₅₅ for the induction of EAE. EAE mice received daily doses of saline, N6022 (1 mg/kg/day/ip), or GSNO (1 mg/kg/day/ip) starting day 9 post-immunization. On day 21, spinal lumbar cords were immunostained for B cells (B220) (FIG. 5A). AMF=anterior median fissure, GM=grey matter, SAS=subarachnoid space, WM=white matter. On day 21, the numbers of B cells (B220⁺), IL-10⁺ B cells, and IL-6⁺ B cells were analyzed in the CNS (spinal cord and Brain) (FIG. 5B) and the spleen (FIG. 5D). The blood levels of IL-10 and IL-6 were also analyzed by ELISA (FIG. 5C). To investigate the role of B cells in T cell-mediated immune responses in EAE, B cells were purified from control (non-EAE) or MOG₃₅₋₅₅-immunized (EAE) mice treated with vehicle or N6022 on day 10. The B cells (2×10⁶ cells) were then transferred to EAE mice (MOG₃₅₋₅₅-immunized) on day 10 and daily clinical score (FIG. 5E-i) and its quantitative summary (area under the curves) (FIG. 5E-ii), blood levels of IL-10 and IL-6 (FIG. 5F-i) and the numbers of Treg (CD4⁺CD25⁺FOXP3^{+/}) and T_{H}17 (CD4⁺IL-17a⁺) cells in the CNS (FIG. 5F-ii) were analyzed. For ELISpot assay of IL-10 producing B cells (FIG. 5G), splenic B cells isolated from control (Ctrl) or EAE (MOG₃₅₋₅₅ immunized; day 21), mice were cultured on IL-10 ELISpot plate coated with MOG₃₅₋₅₅ peptide (10 μg/ml) to detect of MOG specific B cells or anti-mouse B220 Ab (5 μg/ml) to detect total B cells. The cells were cultured in the presence or absence of 50 μM GSNO for 48 hrs. Then, the numbers of MOG-specific B cells or total B cells (B220) producing IL-10 were counted. Data are expressed as mean±standard error mean (SEM). ** p≤0.01, *** p≤0.001 vs. control mice and ⁺p≤0.05, ⁺⁺p≤0.01, ⁺⁺⁺p≤0.001 vs. as indicated (n.s.; not significant). The “n” represents the number of animals/cell cultures used for analyses.

FIGS. 6A-D—Role of GSNO and iNOS in immunomodulation of EAE disease. EAE is induced by MOG₃₅₋₅₅ immunization of C57BL/6 mice and spinal cord expression/activity of GSNOR (FIG. 6A-i) and iNOS (FIG. 6A-ii) were analyzed. C57BL/6 (wt), Gsnor^{-/-}, and iNos^{-/-} mice were immunized by MOG₃₅₋₅₅ for induction of EAE and daily clinical scores and the areas under the curves (AUC) (FIG. 6B-i), myelin status (FIG. 6B-ii), CNS infiltration of CD3⁺ T cells (FIG. 6B-iii) and mononuclear cells (H&E staining) (FIG. 6B-iv) were analyzed. At the peak of disease (day 25 post-immunization), the numbers of Treg cells (CD4⁺CD25⁺Foxp3^{+/-}) and T_{H}17 cells (CD4⁺IL-17a⁺) in the spleen (FIG. 6C-i) and the spinal cord (FIG. 6C-ii) and the levels of IL-10, IL17a, and IL-6 in the spinal cords (FIG. 6C-iii) were analyzed by flow cytometry and ELISA. B cells were purified from MOG₃₅₋₅₅-immunized (EAE) wild type (wt), Gsnor^{-/-}, and iNos^{-/-} mice and the numbers of IL-10⁺ and IL-6⁺ B cells (B220⁺) were analyzed by flow cytometry (FIG. 6D-i). The isolated B cells (2×10⁶ cells) were then transferred to recipient EAE mice (MOG₃₅₋₅₅-immunized/PTX-treated) on day 10 post-immunization and daily clinical score and its quantitative summary (area under the curves) (FIG. 6D-ii), and blood levels of IL-10, IL-6, and IL-17a (FIG. 6D-iii) were analyzed. Data are expressed as mean±SEM. ** p≤0.01, *** p≤0.001 vs. control mice and ⁺p≤0.05, ⁺⁺p≤0.01, ⁺⁺⁺p≤0.001 vs. as indicated (N.S.; not significant). n=number of animals.

FIGS. 7A-B. GSNORi therapy of Spike S1 treated mice improves the body weight and body temperature. Treatment of Spike S1 protein results in increase in body temperature and loss of body wt in response to inflammatory response. GSNORi therapy protects against the Spike S1 protein induced increase in body temperature and body weight.

FIG. 8. T cell response in spleen of GSNO/GSNORi therapy in Spike S1 mice model of COVID disease. Spike S1 protein treatment induced the polarization of Th cells into Th1 and Th17 cells (pro inflammatory activity) but with relatively little effects on Treg cells (anti-inflammatory activity. GSNO or GSNORi therapy down regulated the Spike S1 induced T cell responses with greater effects on Th17 cells. On the other hand, GSNO/GSNORi had practically no effect on Treg cells.

FIG. 9. B cell responses in spleen for GSNO/GSNORi in Spike S1 mice model. Spike S1 treated mice had four-fold increase in IL-6 positive cells (proinflammatory activity) as compared to relatively small increase in IL-10 positive cells (anti-inflammatory activity). Treatments with GSNO/GSNORi decreased the IL-6 positive B cells but increased the IL-10 positive B cells.

FIG. 10. Protection of vascular integrity by GSNO/GSNORi in lungs and brain of Spike S1 mice model. The inventors studied the integrity of blood vessels by following the release of EVANs blue dye from blood vessels, damaged by inflammation and hypoxia induced vascular injury, in lungs and brains as representation of multi organ damage in Spike S1 mice model of COVID-19 disease. EVANs blue was injected into vein of control and Spike Si mice with and without GSNORi therapy. The release of blue dye into tissues seen as blue color in organs represents the degree of inflammation and hypoxia induced vascular damage (integrity) in these tissues of Spike S1 mice (S1). GSNORi therapy (S1+N6022) decreased the vascular damage as decrease in release of blue dye from blood vessels into lung and brain tissues documenting the efficacy of GSNORi for vascular protection. The observed GSNORi protection in both lungs and brains represent that GSNORi therapy will protect the vascular integrity in all organs as multiorgan dysfunctions in COVID-19 disease.

FIGS. 11A-D. Quantitation of EVANs blue and edema in Spike S1 mice model of COVID disease with or with GSNORi (N6022) therapy. FIGS. 11A-D show quantitative protection of Vascular integrity by GSNORi therapy in Spike S1 mice model of COVID disease: Efficacy of GSNORi therapy in Spike (S1) treated mice model was investigated as Quantitation of release of EVANs blue dye (FIG. 11B) and changes in tissue water content (edema) in lung and brain tissues (FIGS. 11C-D). The increased release of blue dye and increase in water content in lungs and brains of Spike 1 treated mice (Spike S1) in FIGS. 11C-D represents inflammation and hypoxia induced vascular damage. GSNORi therapy (Spike S1+N6022) decreased the edema (pneumonia) as well as release of EVANs blue dye representing protection of vascular integrity thus functions in COVID mice model. The observed protection of GSNORi in both lungs and brains represent that GSNORi therapy will protect the vascular integrity in all organs as multiorgan dysfunctions in COVID-19 disease.

### DESCRIPTION OF ILLUSTRATIVE EMBODIMENTS

A rapid and coordinated innate immune response is the first line of defense against viral infection; however, dysregulation and delayed and weak immune response (low levels of antiviral interferons of IFNα/β at early stage) leading to viral infection and subsequent expressions of high levels of proinflammatory cytokines and chemokines, T cell apoptosis leading to dysfunctional immune response as cytokine storm and subsequent disease pathologies. In addition to virus-induced imbalance of innate response, specific/adoptive and autoimmune responses (Th1/Th2/Th17/Treg dysregulations) participate in virus-induced imbalance of immune activities.

SARS-CoV-2 has been shown to invokes a hyper inflammatory state driven by multiple cell types (neutrophils, macrophages, T-cells and B-cells) in innate, adaptive and auto immune responses for induction of specific cytokines. This Imbalanced response of immune system contributes to increased specific cytokine profile (IFNγ, IL-1β, IL-6/IL-4, IL-10/IL-17, IL-6, IL-33/IL-37) that play critical role, as cytokine storm in inducing ARDS and subsequent global hypoxia and related vascular dysfunctions and venous and arterial thromboembolic complications. IL-6 expression is high in SARS-CoV-2 patients and it acts as a key driver of the hyper inflammation associated with SARS-CoV-2 and is reported to be a low survival predictor among patients with acute respiratory distress syndrome (Heinrich et al., Biochem J. 1990; 265: 621-636). IL-6 and TNFα were also reported to be associated with T cell depletion/dysfunction in SARS-CoV-2 patients and levels of these cytokines are inversely proportional to Tcell population in patients in intensive care units (McGonagle et al., Autoimmune Rev. 2010: 19(6):102527). IL-6 plays a central role in acute inflammation produced by immune (innate, adaptive and auto) systems via IL6R/gp130 receptor activated JAK/STAT and RAS/RAF signaling pathways regulating cell proliferation, differentiation, oxidative stress and immune regulation. In addition to immune cell dysfunctions, the increased levels of IL-6 induce unremitting fever, hyperferritinemia in SARS-CoV-2 related pathologies affecting multiple organ systems. Significant (2.9-fold) increased levels of IL-6 in patients with SARS-Cov-2 suggest a potential role of drugs targeting IL-6 (inhibitors) in preventing damage to lung tissue caused by uncontrolled cytokine release syndrome (cytokine storm) in SARS-CoV-2 patients.

Therefore, inventors believe an ideal drug for treating SARS-Cov-2 needs to have dual function: down regulation of proinflammatory mechanisms while upregulating the anti-inflammatory mechanisms. Present day drugs including antibodies targeting IL-6/IL-6R, Jak inhibitor and corticosteroids are limited in this regard. Here, the inventors provide experimental evidence that GSNO/GSNORi-mediated mechanisms generate such dual activity by down regulation of proinflammatory immune mechanisms, including decreased expression of IL-6, as well as upregulation of anti-inflammatory immune mechanisms including increased expression of IL-10. These and other aspects of this disclosure are set out in detail below.

### I. DISEASE STATES

A. Viruses

In accordance with the present disclosure, it is proposed to treat viral disease using GSNO/GSNOR inhibitors. In particular, viral diseases that induce hyperinflammatory reactions, such as those that impact the pulmonary system, are of particular interest. Viruses contemplated for treatment include ebolavirus, hantavirus, SARS-CoV-1, SARS-CoV-2, MERS-CoV, influenza virus, Dengue virus, and respiratory syncytial virus.

SARS-CoV-2.

Coronaviruses are positive-sense single stranded RNA viruses with genome between 26.2 and 31.7 kb RNA and sequence analysis of SARS-CoV-2 revealed 96% homology with bat virus and Codes for 16 non-structural and 4 major structural proteins Structural proteins (spike (S) protein, membrane (M) protein, nucleocapsid (N) protein and the nucleocapsid (E) protein) required to produce the virus particle (Schoeman et al., Virol J 16:69, 2019). The S (SPIKE) glycoprotein mediates SARS-Cov-2 entry into cell following it's binding to the angiotensin converting enzyme (ACE2) (Hoffman et al., Cell 2020.02.052). ACE2 is highly expressed on the cell surface of many tissues/organs including lungs and lower tract of the respiratory system whereas the common flu virus binds to the upper air way tract. Therefore, differential expression of ACE2 may potentially explain the basis of serious respiratory insufficiency in SARS-Cov-2 disease (Zheng et al., Nat, Rev. Cardiol. 10.1038, 2020).

Patients with SARS-CoV-2 show clinical manifestations of fever, nonproductive cough, dyspnea, myalgia, fatigue and radiographic evidence of pneumonia and further complication based on individual risk factors (Li et al., J. Pharmaceutical Analysis 10: 102-108, 2020). Clinically disease caused by SARS-CoV-2 can be classified as a three phase disease, even though multiple disease mechanisms participate in the respective disease phases; Phase 1, viral infection and its replication, Phase 2 is virus induced immune dysfunction leading to the so called “cytokine storm” and associated respiratory failure. Phase 3 is cytokine storm and respiratory failure-induced acute respiratory distress syndrome (ARDS) and global hypoxia associated vascular/dysfunctions and multiple organ failure.

So far, a number of small studies have reported efficacy of anti-IL-6 agents (Tocilizumab) for SARS-CoV-2 patients (Lou et al., J. Med. Virol. 2020; 92: 814-818., Giambenedetto et al., J. Med. Virol. 2020.doi 10.1002/jmv.25897., Michot et al., Ann. Oncol. 2020; 961-964, Ferrey et al., Am. J. Nephrol. 2020; 28: 1-6., Mihai et al., Ann. Rheum. Dis. 2020; 79:668-669). Presently, there are a number of ongoing randomized multicenter clinical trials for IL-6 inhibitors; 8 studies with tocilizumab, 5 studies with Sarilumab and 2 with siltuimab (Pozo et al., Eur. Rev. Med. Pharmcol. Sci. 2020; 24: 7475-7478; Hashizume, M. Inflamm. Regen. 2020; 40: 24-8., Atal, S. and Fatima, Z. and Crisafulli et al. BioDrugs 2020).

Tocilizumab or “TCZ” is an IL6 receptor antibody that bocks IL-6 signaling and thus blocks the IL-6 mediated inflammatory mechanisms. It is widely used in rheumatoid arthritis patients and has been approved for life threatening cytokine storm syndrome caused by chimeric antigen receptor T-cell (CART) immunotherapy. TCZ use for SARS-CoV-2 patients was found to be effective in reducing fever within a few days and 75% patients had improved oxygenation. The optical lung lesion on CT scan resolved in 90.5% of patients. The lymphocyte counts also returned to normal in 53% patients.

Remdesivir, an analog of adenosine targeting RNA polymerase to inhibit RNA synthesis thus viral replication. A recent study investigation with SARS-CoV-2 patients reported limited efficacy in reducing the disease duration but without affecting mortality. Chloroquine and hydroxychloroquine are prophylactic drugs for malaria and are also being used of autoimmune disease such as rheumatoid arthritis and lupus. Based on initial investigation these anti-malarial drugs were expected to have therapeutic potential for SARS-CoV-2 patients. However, subsequent studies did not support the earlier findings leading to FDA based restriction on use of chloroquine for SARS-CoV-2 patients especially patients with impaired immune system.

There are other drugs presently under consideration for SARS-Cov-2 therapy. For example, JAK inhibitors like Inhibitor of AP2 associated protein kinase 1 (AAK1) are known to regulate virus endocytosis thus entry of SARS-CoV-2 into cells. The JAK inhibitor Baricitinib can also inhibit AAK1. However, the limitation of JAK inhibitors is that it can also inhibit inflammatory response for IFNα and TNFα, IFNγ expressions. These cytokines participate in antiviral activity and thus warrant safety concerns. Clinical trials of Jakotinib in SARS-CoV-2 patients (ChiCTR200030170) and in combination with mesenchymal cells (ChiCTR2000029580) are ongoing at present.

Over the years a number of trials were conducted on patients with different corona family members (SARS, MERS) using corticoids but there is no conclusive evidence that glucocorticoid therapy is effective (Auyeung et al., J Infect. 51:98-102, 2005; Yam et al., J. Infect. 54:28-39, 2007). Similarly, use of glucocorticoids for SARS-Cov-2 patients was reported to be ineffective (Russell et al., Lancet 395: 473-475, 2020). Accordingly, WHO does not support the use of corticoids for SARS-CoV-2 patients.

A recent study reported a word of caution to treat SARS-CoV-2 patients with imbalanced immune activities (IFNγ, ILβb/IL-4, IL-10/IL-17, IL-6, IL-33/IL-37), known as cytokine storm, with immune-suppressive or immune-simulating drugs is likely to cause high risk of developing severe and even fatal respiratory disease (Russel et al., ecancer 2020, 15; world-wide-web at ecancer.org; doi.org/10.3332/ecance.2020.1022).

B. Other Disease States

Acute and Chronic Inflammatory Disease Conditions.

IL-6 is traditionally considered to regulate acute phase disease conditions but recent observations of increased IL-6 expressions in chronic disease conditions of autoimmune disorders and in various cancers point to its role in chronic disease pathologies (Ivashkiv et al., Nat Rev. Immunol. 7(6) 429-442, 2007). During inflammatory episode, IL-6 is highly expressed, ranging from normal levels of 1-5 pg/ml to 1000 ug/ml, in various disease conditions (Wage et al., I. Exp. Med. 169 (10 333-338, 1989). IL-6 activates the cells by first binding to its receptor (IL-6 R) that dimerizes with gp130. A hexamer complex of IL-6/IL-6R/gp130 leads to intracellular activation of receptor associated kinases (JAK1, JAK2 and Tyk2) which in turn phosphorylates STAT1/STAT3 for downstream signaling. In addition to IL-6 signaling through gp130, it also transmits signaling for other cytokines (IL-11, IL-2), oncostatin, ciliary neurotrophic factor, cardiotrophin and leukemia inhibitory factor for respective activities (Jones et al., J. Clin. Med. Invest. 121 (9), 2011). Further IL-6 induced differentiation of TH17 and expression of IL-17 underscore its role in disease pathologies associated IL-6/STAT3 signaling pathways. Multiple Cytokines (IFN-γ, GM-CSF, IL6, IL10, IL-15, IL-23) signal through JAK/STAT pathway. Major challenge is to delineate how specific cytokines interfere with specific inflammatory processes to affect the disease outcome.

IL-6 is an essential cytokine that transmits signals in response to pathogen invasion or tissue damage to stimulate acute phase reactions and immune responses. However, excessive and sustained production of IL-6 is associated with various inflammatory disease pathologies. As IL-6 is involved in various diseases, therapeutic approaches to block IL-6 signaling have been developed, such as antibodies to IL-6 and IL-6 receptor (tocilizumab; TCZ) (Narazaki and Kishimoto, Int. Mol. Sci. 19, 3528; doi.3390/ijms 19113528). TCZ, a humanized anti-IL-6R, inhibits IL-6 binding to the IL-6R and its treatment provides efficacy for patients with Neuromyelitis optica, Castleman's disease, Crohn's disease/inflammatory bowel disease and Cytokine Releasing Syndrome and multorgan failure (Narazaki and Kishimoto, Int. Mol. Sci. 19, 3528; doi.3390/ijms 19113528). Our studies reporting the inhibition of IL-6 expression by GSNO/GSNOR inhibitor (GSNORi) indicate potential efficacy of GSNOR inhibitors in IL-6 induced above disease conditions including progressive MS(Yao et al., Pharmacol. & Ther. 141: 125-139, 2014 and steroid sparing rheumatoid arthritis, giant cell arthritis, systemic onset juvenile idiopathic arthritis and systemic sclerosis.

Cancer. Stat3 activity often correlates with tumorigenesis, tumor growth, survival, angiogenesis and metastatic processes of many cancers including colorectal cancer, head and neck cancer, pancreatic cancer, breast and prostate cancer (Yu et al., Nat. Rev. Cancer (9(11) 798-809, 2009; Grivennikov et al., Cancer Cell 16(2): 103-113, 2009). High levels of IL-6 in patients with breast cancer, lung and hematopoietic tumor correlated with poor clinical prognosis (Bachelot et al., Br. J. Cancer 88(11):1721-1726, 2003; Hong et al., Cancer 110(9) 1911-1928, 20o7). Based on our studies documenting the inhibition of IL-6 expression thus inhibition of IL-6 induced cellular signaling pathway by GSNO reductase inhibitors (GSNORi) can be of therapeutic potential as a drug in IL-6 associated cancer pathology and cancers.

### II. METHODS OF USE

Embodiments of the present disclosure concern methods of treating SARS-CoV-2 infection and disease by administering an effective amount of GSNO and/or one or more GSNO reductase inhibitors to a subject.

S-Nitrosoglutathione (GSNO) is an endogenous S-nitrosothiol (SNO) that plays a critical role in nitric oxide (NO) signaling and is a source of bioavailable NO. The enzyme GSNO reductase (GSNOR) reduces S-nitrosoglutathione (GSNO) to an unstable intermediate, S-hydroxylaminoglutathione, which then rearranges to form glutathione sulfonamide, or in the presence of GSH, forms oxidized glutathione (GSSG) and hydroxylamine. Through this catabolic process, GSNOR regulates the cellular concentrations of GSNO and plays a central role in regulating the levels of endogenous S-nitrosothiols and controlling protein S-nitrosylation-based signaling. S-Nitrosoglutathione reductase (GSNOR) regulates S-nitrosothiols (SNOs) and nitric oxide (NO) in vivo through catabolism of S-nitrosoglutathione (GSNO). GSNOR and the anti-inflammatory and smooth muscle relaxant activities of SNOs, GSNO, and NO play significant roles in pulmonary, cardiovascular, and gastrointestinal function.

In some aspects, a subject is administered an inhibitor of GSNO reductase (GSNOR). For example, N6022 is a potent and reversible GSNO reductase inhibitor that may be used in the methods of the present disclosure (Sun et al., Bioorg Med Chem Lett. 21(19):5849-53, 2011; Green et al., Biochemistry 51(10):2157-68, 2012; both incorporated herein by reference). Further GSNO reductase inhibitors that may be used in the present disclosure include, but are not limited to, substituted pyrrole analogs (e.g., described in U.S. Pat. No. 8,642,628; incorporated herein by reference) and chromone inhibitors of GSNOR, such as 4-(2-(difluoromethyl)-7-hydroxy-4-oxo-4H-chromen-3-yl)benzoic acid, as disclosed in U.S. Pat. No. 8,669,381; incorporated herein by reference.

It is contemplated that the GSNO and/or at least one GSNO reductase inhibitor may be administered in combination with one or more additional therapies. The additional therapies may comprise anti-inflammatories or other SARS-CoV-2 therapies mentioned above.

As used herein, “treating” describes the management and care of a patient for the purpose of combating a disease, condition, or disorder and includes the administration of a compound of the present invention to prevent the onset of the symptoms or complications, alleviating the symptoms or complications, or eliminating the disease, condition or disorder. More specifically, “treating” includes reversing, attenuating, alleviating, minimizing, suppressing or halting at least one deleterious symptom or effect of a disease (disorder) state, disease progression, disease causative agent (e.g., bacteria or viruses), or other abnormal condition. Treatment is continued as long as symptoms and/or pathology ameliorate.

The patient can be any animal, domestic, livestock or wild, including, but not limited to cats, dogs, horses, pigs and cattle, and preferably human patients. As used herein, the terms patient and subject may be used interchangeably.

The GSNO or GSNO reductase inhibitors can be utilized in any pharmaceutically acceptable dosage form, including but not limited to injectable dosage forms, liquid dispersions, gels, aerosols, ointments, creams, lyophilized formulations, dry powders, tablets, capsules, controlled release formulations, fast melt formulations, delayed release formulations, extended release formulations, pulsatile release formulations, mixed immediate release and controlled release formulations, etc. Specifically, the GSNO reductase inhibitors described herein can be formulated: (a) for administration selected from the group consisting of oral, pulmonary, intravenous, intra-arterial, intrathecal, intra-articular, rectal, ophthalmic, colonic, parenteral, intracisternal, intravaginal, intraperitoneal, local, buccal, nasal, and topical administration; (b) into a dosage form selected from the group consisting of liquid dispersions, gels, aerosols, ointments, creams, tablets, sachets and capsules; (c) into a dosage form selected from the group consisting of lyophilized formulations, dry powders, fast melt formulations, controlled release formulations, delayed release formulations, extended release formulations, pulsatile release formulations, and mixed immediate release and controlled release formulations; or (d) any combination thereof.

Oral compositions generally include an inert diluent or an edible carrier. They can be enclosed, for example, in gelatin capsules or compressed into tablets. For the purpose of oral therapeutic administration, the GSNOR inhibitor can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition.

It is especially advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of GSNO or GSNOR inhibitor calculated to produce the desired therapeutic effect in association with the required pharmaceutical carrier. The specification for the dosage unit forms of the invention are dictated by and directly dependent on the unique characteristics of the GSNO or GSNOR inhibitor and the particular therapeutic effect to be achieved, and the limitations inherent in the art of compounding such an active agent for the treatment of individuals.

Pharmaceutical compositions according to the present disclosure comprising GSNO and/or at least one GSNOR inhibitor can comprise one or more pharmaceutical excipients. Examples of such excipients include, but are not limited to binding agents, filling agents, lubricating agents, suspending agents, sweeteners, flavoring agents, preservatives, buffers, wetting agents, disintegrants, effervescent agents, and other excipients. Such excipients are known in the art. Exemplary excipients include: (1) binding agents which include various celluloses and cross-linked polyvinylpyrrolidone, microcrystalline cellulose, such as Avicel® PH101 and Avicel® PH102, silicified microcrystalline cellulose (ProSolv SMCC™), gum tragacanth and gelatin; (2) filling agents such as various starches, lactose, lactose monohydrate, and lactose anhydrous; (3) disintegrating agents such as alginic acid, Primogel, corn starch, lightly crosslinked polyvinyl pyrrolidone, potato starch, maize starch, and modified starches, croscarmellose sodium, cross-povidone, sodium starch glycolate, and mixtures thereof; (4) lubricants, including agents that act on the flowability of a powder to be compressed, include magnesium stearate, colloidal silicon dioxide, such as Aerosil® 200, talc, stearic acid, calcium stearate, and silica gel; (5) glidants such as colloidal silicon dioxide; (6) preservatives, such as potassium sorbate, methylparaben, propylparaben, benzoic acid and its salts, other esters of parahydroxybenzoic acid such as butylparaben, alcohols such as ethyl or benzyl alcohol, phenolic compounds such as phenol, or quaternary compounds such as benzalkonium chloride; (7) diluents such as pharmaceutically acceptable inert fillers, such as microcrystalline cellulose, lactose, dibasic calcium phosphate, saccharides, and/or mixtures of any of the foregoing; examples of diluents include microcrystalline cellulose, such as Avicel® PH101 and Avicel® PH102; lactose such as lactose monohydrate, lactose anhydrous, and Pharmatose® DCL21; dibasic calcium phosphate such as Emcompress®; mannitol; starch; sorbitol; sucrose; and glucose; (8) sweetening agents, including any natural or artificial sweetener, such as sucrose, saccharin sucrose, xylitol, sodium saccharin, cyclamate, aspartame, and acesulfame; (9) flavoring agents, such as peppermint, methyl salicylate, orange flavoring, Magnasweet® (trademark of MAFCO), bubble gum flavor, fruit flavors, and the like; and (10) effervescent agents, including effervescent couples such as an organic acid and a carbonate or bicarbonate. Suitable organic acids include, for example, citric, tartaric, malic, fumaric, adipic, succinic, and alginic acids and anhydrides and acid salts. Suitable carbonates and bicarbonates include, for example, sodium carbonate, sodium bicarbonate, potassium carbonate, potassium bicarbonate, magnesium carbonate, sodium glycine carbonate, L-lysine carbonate, and arginine carbonate.

The phrases “pharmaceutical or pharmacologically acceptable” refers to molecular entities and compositions that do not produce an adverse, allergic, or other untoward reaction when administered to an animal, such as a human, as appropriate. The preparation of a pharmaceutical composition comprising an antibody or additional active ingredient will be known to those of skill in the art in light of the present disclosure. Moreover, for animal (e.g., human) administration, it will be understood that preparations should meet sterility, pyrogenicity, general safety, and purity standards as required by FDA Office of Biological Standards.

As used herein, “pharmaceutically acceptable carrier” includes any and all aqueous solvents (e.g., water, alcoholic/aqueous solutions, saline solutions, parenteral vehicles, such as sodium chloride, Ringer's dextrose, etc.), non-aqueous solvents (e.g., propylene glycol, polyethylene glycol, vegetable oil, and injectable organic esters, such as ethyloleate), dispersion media, coatings, surfactants, antioxidants, preservatives (e.g., antibacterial or antifungal agents, anti-oxidants, chelating agents, and inert gases), isotonic agents, absorption delaying agents, salts, drugs, drug stabilizers, gels, binders, excipients, disintegration agents, lubricants, sweetening agents, flavoring agents, dyes, fluid and nutrient replenishers, such like materials and combinations thereof, as would be known to one of ordinary skill in the art. The pH and exact concentration of the various components in a pharmaceutical composition are adjusted according to well-known parameters.

The term “therapeutic benefit” or “therapeutically effective” as used throughout this application refers to anything that promotes or enhances the well-being of the subject with respect to the medical treatment of this condition. This includes, but is not limited to, a reduction in the frequency or severity of the signs or symptoms of a disease.

### III. EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1—Overview

The inventors findings show that GSNO (S-nitosoglutathione) and GSNORi regulate the immune (innate, adaptive and autoimmune) dysfunctions via down regulation of proinflammatory response including IL-6 while upregulating the anti-inflammatory response including IL-10.

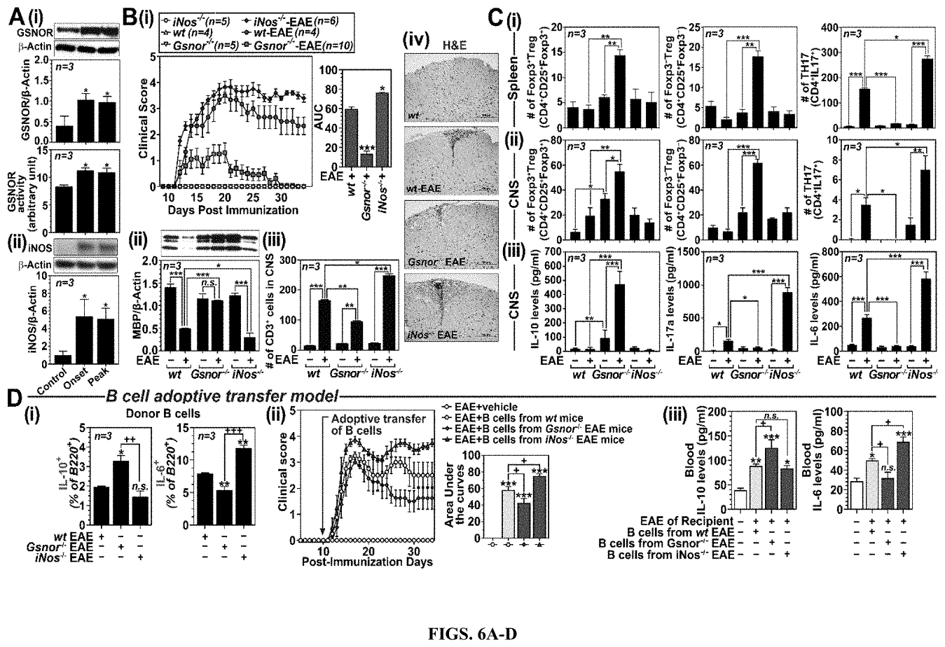

GSNO (S-nitrosylated glutathione) or GSNORi function to regulate the cellular homeostasis of GSNO, a physiological human body component. It is synthesized by redox based reaction between GSH (glutathione) and NO (nitric oxide) and is catabolized by GSNOR in the cells. In recent past, GSNO mediated protein S-nitrosylation has been recognized to regulate various cellular signaling thus functions. Under inflammatory and hypoxic conditions (Covid-19 disease conditions) the increased cellular levels GSNOR (FIGS. 6A-D) decrease the cellular homeostasis of GSNO and thus loss of GSNO mediated cellular regulations including immune functions. Exogenous supplementations of GSNO or upregulation of cellular GSNO by inhibiting its degradation using GSNORi. Immunological disease of SARS-CoV-2 involves the participation of cell types of innate, adaptive as well as autoimmune activities (Rodriguez et al. Journal of Autoimmunity, 114: 102506, 2020). The inventors report that GSNO and GSNORi target the SARS-CoV-2 induced immune (innate, adaptive and autoimmune) dysfunctions.

### Example 2—Results

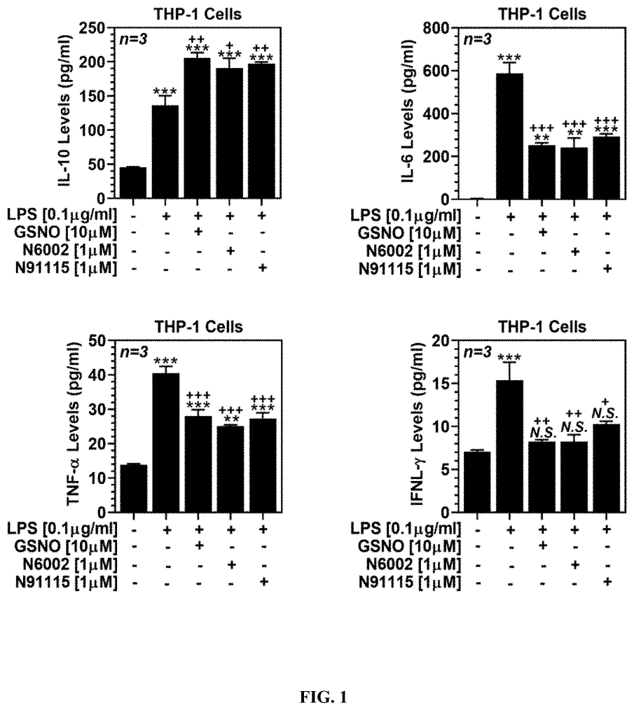

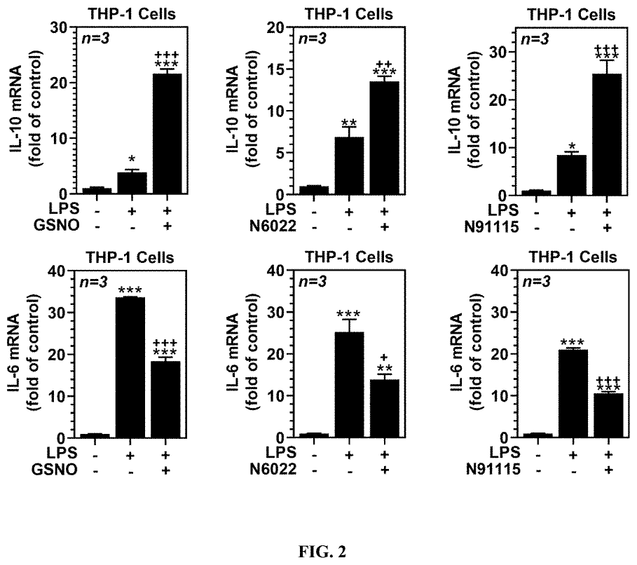

Macrophages are the major source of IL-6 among the innate immune system. Using human macrophage sell lone of THP-1, FIG. 1 shows the dual function of GSNO/GSNORi in innate immunity. GSNO as well as inhibitors of GSNOR (V6002 and N9111) inhibit the expression of proinflammatory cytokines (TNFα, IFNγ and IL-6) in LPS stimulated THP-1 cells, a human macrophage cell line. On the other hand, treatment of LPS stimulated THP-1 cells with GSNO or GSNORi (N6002 and N91115) also induced the expression of anti-inflammatory cytokine IL-10.

FIG. 2 shows that treatment pf LPS stimulated THP-1 human macrophage cell line with GSNO or GSNORi (N6022, N91115 down regulate the mRNA levels of IL-6 while upregulating the mRNA levels of IL-10. The data in FIGS. 1-2 documents that GSNO and GSNORi regulate the innate immune activity via down regulation of expression proi-inflammatory IL-6 cytokine while upregulating the anti-inflammatory IL-10 cytokine.

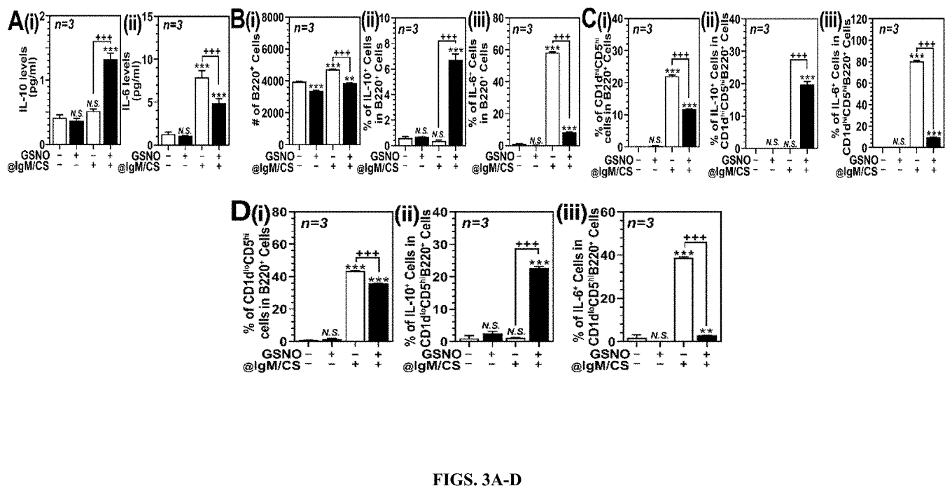

In adaptive immunity, B cells are the major source of IL-6. FIGS. 3A-D show the direct effect of GSNO and GSNORi on the B cell expression of IL-10 versus IL-6, naïve B cells stimulated with anti-IgM Ab (@IgM: for activation of BCR) and co-stimulated (CS) with recombinant BAFF (rBAFF) and anti-CD40 Ab in the presence or absence of GSNO (see legend of FIGS. 3A-D for experimental detail). FIG. 3A shows that the stimulation/co-stimulation of B cells had no effect on IL-10 production but induced IL-6 production. GSNO treatment did not affect IL-10 and IL-6 production under the quiescent conditions but did increase IL-10 production and decrease IL-6 production under stimulatory/co-stimulatory conditions (FIG. 3A). GSNO treatment slightly decreased the number of total B cells (B220⁺) under both quiescent and stimulatory/costimulatory conditions (FIG. 3B-i). However, it drastically increased IL-10⁺ B cells, while decreasing the IL-6⁺ B cells, under stimulatory/costimulatory conditions (FIGS. 3B-ii and -iii). Activated B cells (CD5^{hi 96}) expressing high levels of CD1d are reported to produce high levels of IL-10 (so-called Breg). FIG. 3C-i and FIG. 3c-iii show that the stimulation/co-stimulation of B cells increased the numbers of total and IL-6⁺ CD1d^{hi}CD5^{hi} B cells and which were decreased by GSNO treatment. The number of IL-10⁺ CD1d^{hi}CD5^{hi} B cells were not increased by stimulation/co-stimulation of B cells in the absence of GSNO but drastically increased in the presence of GSNO (FIG. 3C-ii). Interestingly, similar observations were also made in CD1d^{lo}CD5^{hi} B cells (FIG. 3D) where GSNO treatment increased the number of IL-10⁺ cells while decreasing the IL-6⁺ cells under stimulatory/costimulatory conditions, thus suggesting that GSNO mediated induction of IL-10 production and suppression of IL-6 production is not limited to Breg but relevant to other subtypes of B cells as well.

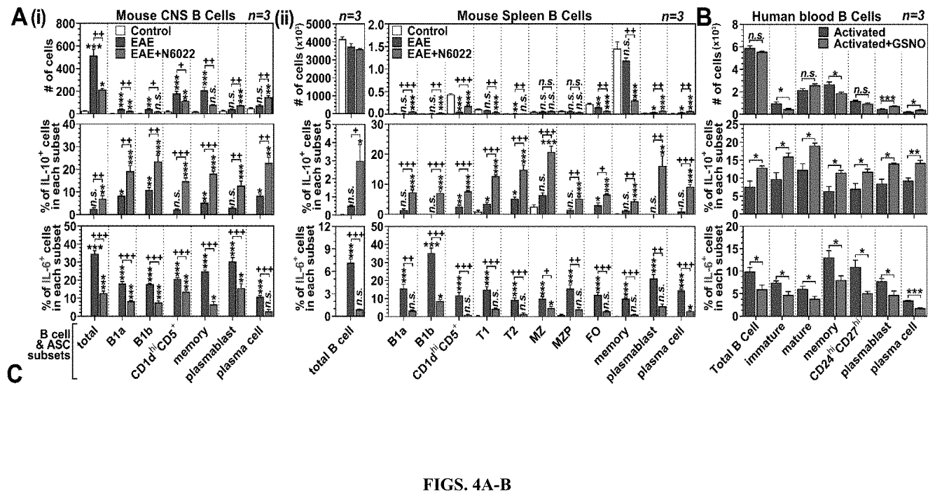

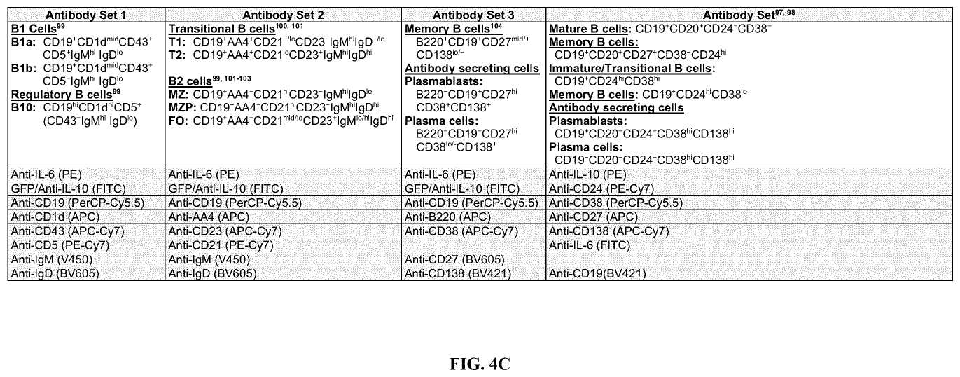

FIGS. 4A-C show GSNO/GSNORi-mediated regulation of IL-10 versus IL-6 in B cell/ASC subsets. Next, the inventors characterized B cell and ASC subsets expressing higher levels of IL-10 and lower levels of IL-6 in response to GSNO/N6022 in the EAE model and in the human blood B cell culture model. EAE induction had no obvious effect on the number of total splenic B cells but increased the numbers of B cells and ASCs in the CNS and which were decreased by N6022 treatment (FIGS. 4A-B). Accordingly, N6022 treatment decreased the numbers of the most subsets of B cells and ASCs in the CNS. In the spleen, N6022 treatment of EAE mice decreased the numbers of several subsets of B cells (e.g., Breg and memory B cells) but the most dramatic reduction was observed in memory B cells. On the other hand, N6022 treatment increased the splenic ASCs (plasmablasts and plasma cells). As expected, the inventors observed that N6022 treatment increased the frequency of IL-10⁺ cells and decreased the frequency of IL-6⁺ cells in almost all B cell and ASC subsets in the CNS as well as the spleen. These data indicate that GSNORi-mediated induction of IL-10 and suppression of IL-6 is not limited to Breg (e.g., CD1d^{hi}CD5⁺) but to all subsets of B cells and ASCs. Next, the inventors investigated the effects of GSNO on subset specific expression of IL-10 versus IL-6 in human B cells stimulated with anti-human IgM/IgG Ab and costimulated with rBAFF and anti-CD40 Ab (FIG. 4B). GSNO treatment had no obvious effect on the number of total B cells but decreased the numbers of some subsets of B cells (e.g., immature B cells and memory B cells) while increasing the numbers of ASCs (plasmablasts and plasma cells). Similar to the EAE mice, GSNO treatment increased the frequency of IL-10⁺ cells but decreased IL-6⁺ cells not only in B10/Breg subset-specific cells (CD24^{hi}CD27⁺ B cells and CD24^{hi} CD38^{hi} immature B cells) but also in all subsets of B cells and ASCs. These in vivo and in vitro data document that GSNO/GSNORi-mediated induction of IL-10 and suppression of IL-6 is not limited to human B10/Breg but to all subsets of B cells and ASCs.

Regulation of Autoimmunity by GSNO and GSNORi.

SARS-COV-2 disease shares inflammatory immune responses with autoinflammatory and autoimmune conditions. Critical SARS-CoV-2 patients have been reported to develop autoantibodies and autoimmune phenomenon at the end-stage of the disease representing autoimmune disorders of Kawasaki disease, Guillain-Barre syndrome and autoimmune cytopenia (Rodriguez et al., Journal of Autoimmunity 114, 102506, 2020). Based on serological, radiological histomorphological similarities, the authors suggest that SARS-COV-2 infection might trigger or stimulate an organ specific autoimmunity in predisposed patient. A study of patients with SARS-COV-2 pneumonia, the authors showed a prevalence of between 20-50% of autoimmune related antibodies (Zhou et al., Clin. Trans. Sci. 2020 doi: 10.111/CTS. 12805). Additionally, patients on immunosuppressive medications are at an increased risk for complications with SARS-COV-2 infection, and many of the currently proposed SARS-COV-2 therapies can interact with these medications. A number of pathogenic viruses are reported to trigger and initiate autoimmunity including parvovirus B19, Epstein Bar-virus (EBV), Cytomegalovirus (CMV), Hepatitis A, C and Rubella virus have been implicated to triggering and initiation of autoimmune diseases including Rheumatoid arthritis, systemic lupus erythematosus and multiple sclerosis (MS) just to name few (Ehrenfeld et al., COVI-19 and autoimmunity, Autoimmune Rev. 19 (8), 102597, 2020).

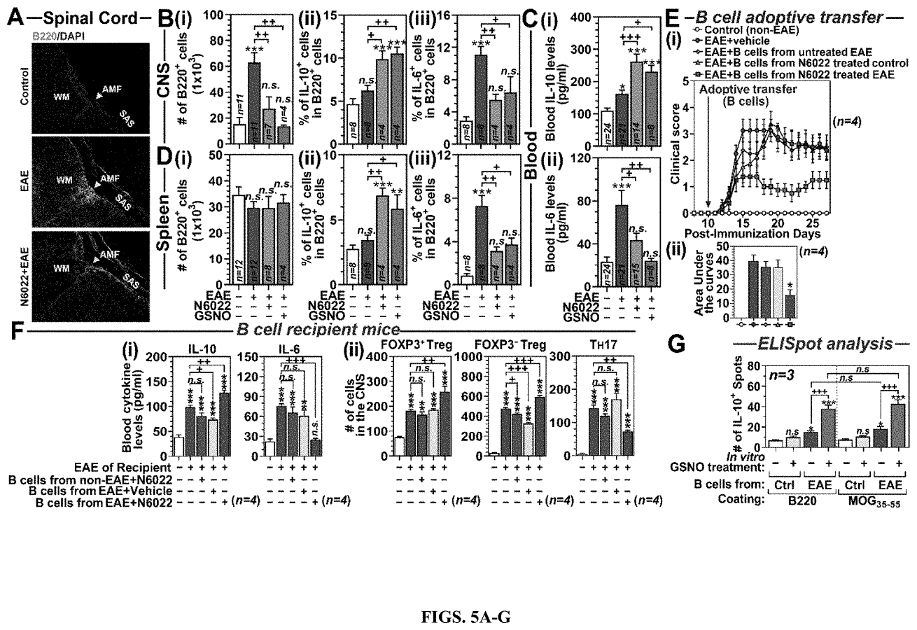

Therefore, the inventors investigated the efficacy of GSNO and GSNORi (N6022) in B cell mediated pathogenesis in autoimmune disorder of EAE, animal model of MS. They explored the effects of N6022/GSNO on B cell activity in EAE. FIGS. 5A and 5B-i show that treatment of EAE mice with N6022/GSNO decreased the CNS infiltration of B cells, especially anterior median fisher (AMF) of the spinal cord. B cells have been recognized as a major source of IL-10 and IL-6 in the blood in MS and EAE. EAE mice had increased blood IL-10 and IL-6 levels and treatment of these mice with N6022/GSNO further increased the IL-10 levels but decreased the IL-6 levels (FIG. 5C). In addition, N6022 (or GSNO) treatment increased the numbers of IL-10⁺ B cells but decreased the number of IL-6⁺ B cells in the CNS and the spleen of EAE animals (FIGS. 5C-D; see the legend of FIGS. 5A-G for the experimental detail).

To further investigate the role of B cells in T cell-mediated immune response in N6022-treated EAE mice, B cells were purified from control, EAE, or N6022-treated EAE mice and transferred to EAE mice on day 10 post-immunization. FIG. 5E shows that only B cells transferred from N6022-treated EAE mice ameliorated the clinical disease of recipient EAE mice. Accordingly, B cell recipients from N6022-treated EAE mice had increased blood IL-10 levels (FIG. 5F-i) and the number of Treg cells in the CNS (FIG. 5F-ii), while they had decreased blood IL-6 levels and T_{H}17 cells in the CNS. These data document the role of B cells in N6022/GSNO-mediated T cell regulation (Treg>T_{H}17) and thus EAE disease. Interestingly, B cell recipients from N6022-treated non-EAE mice had no disease attenuation, no significant alterations in the blood cytokine levels as well as CNS Treg and T_{H}17 cells, suggesting that the observed B cell-mediated regulation of Treg and T_{H}17 cells in EAE mice is an autoantigen (MOG)- or disease-specific. Therefore, the inventors examined the MOG₃₅₋₅₅-specific B cell production of IL-10 in response to the GSNO treatment by enzyme-linked immune absorbent spot (ELISpot) assay. FIG. 5G shows that GSNO treatment of B cells from control mice (non-EA) did not increase the number of IL-10⁺ B cells. However, GSNO treatment of B cells from EAE mice increased the number of IL-10⁺ B cells. Interestingly, GSNO treatment increased the numbers of IL-10 producing B cells in similar ranges in the anti-B220 mAb coated plate and in the MOG₃₅₋₅₅ peptide coated plate, thus indicating that B cells expressing IL-10 are mostly MOG₃₅₋₅₅ specific.

Role of GSNOR/GSNO in B Cell-Mediated Immunopathogenesis of Autoimmune Disease of EAE.

EAE disease involves increased CNS expression of iNOS, as well as increased expression/activity of GSNOR (FIG. 6A), key players in the synthesis and degradation of GSNO. To assess the roles of GSNOR and iNOS in autoimmune disease and pathogenesis of EAE, EAE was induced in C57BL/6 (wt), Gsnor^{-/-}, and iNos^{-/-} mice. As expected, Gsnor^{-/-} mice developed very mild EAE (FIG. 6B-i) with mild degrees of demyelination (FIG. 6B-ii) and less inflammatory cell infiltration (FIG. 6B-iii and FIG. 6b-iv). However, iNos^{-/-} mice developed very severe EAE with higher degrees of demyelination and CNS infiltration of immunocytes compared to wt mice. Consistent with N6022 treated mice above, Gsnor^{-/-} mice had lower T_{H}17/IL-17a immune responses but higher Treg/IL-10 immune responses in the spleens (FIG. 6C-i) and the CNS (FIGS. 6C-ii) as compared to wt EAE mice. Conversely, iNos^{-/-} EAE mice showed opposing results. These studies document that cellular GSNO homeostasis is important in subset specific immune modulation of autoimmune disease of EAE (T_{H}17 vs. Treg) and thus identifies it as a novel target and potential treatment option for autoimmune disease of MS/EAE. FIG. 6D shows that MOG₃₅₋₅₅-immunized Gsnor^{-/-} mice had a greater number of IL-10⁺ B cells and fewer number of IL-6⁺ B cells as compared to MOG₃₅₋₅₅-immunized wt mice while MOG₃₅₋₅₅-immunized iNos^{-/-} mice showed opposing results. Accordingly, the adoptive transfer of B cells from MOG₃₅₋₅₅-immunized Gsnor^{-/-} mice ameliorated EAE disease of recipient EAE mice (FIG. 6D-ii) with increased levels of IL-10 and decreased levels of IL-6 in the blood (FIG. 6D-iii), while the adoptive transfer of B cells from MOG₃₅₋₅₅-immunized iNos—mice showed opposing results. These data documents the role of B cells in GSNOR/iNOS-mediated T cell regulation (Treg vs. T_{H}17) and progression of autoimmune disease of EAE.

Efficacy of GSNO/GSNORi Treatment in COVID Spike S-1 Protein Induced Immune Dysfunction Mice Model.

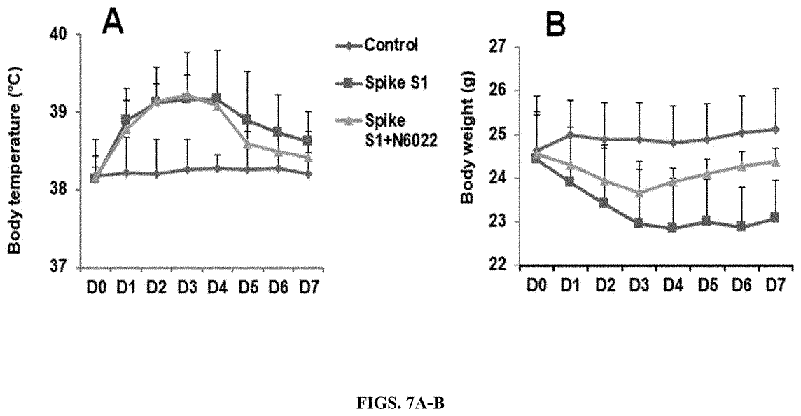

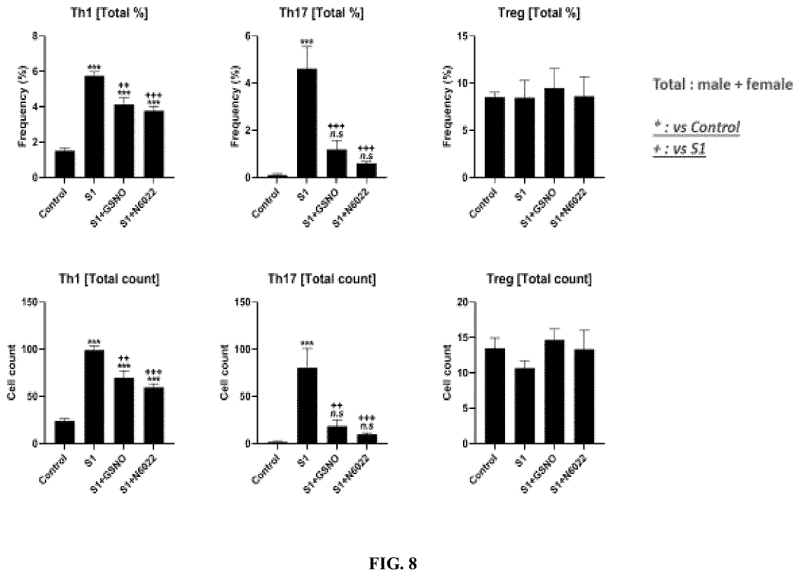

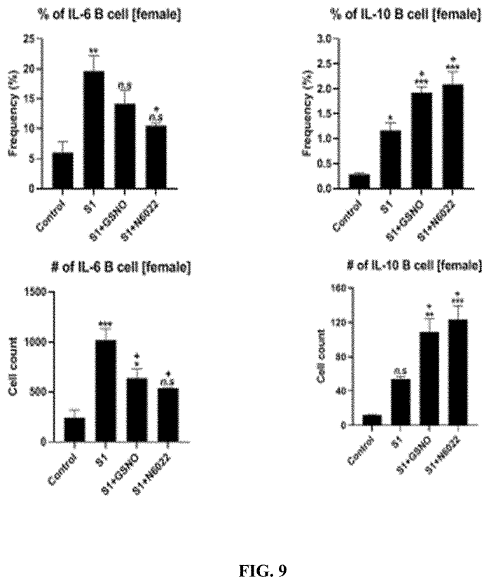

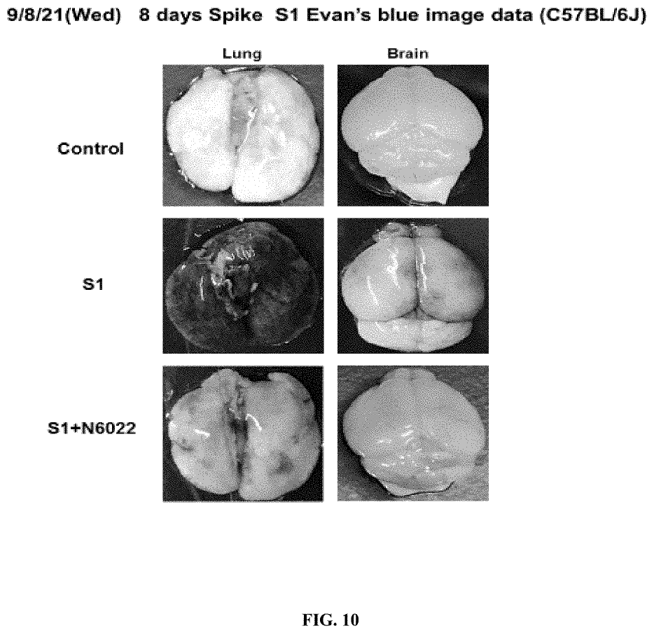

To study the efficacy of GSNO/GSNORi on COVID-19 induced immune dysfunction, we used COVID-19 Spike S-1 protein mice model as described previously (PMID: 33426604). In this model recombinant spike 1 protein dissolved in saline was delivered to each nostril of C57/BL6 mice and control mice received the same volume of saline. Spike S-1 treated mice were divided into three groups; GSNO treated, GSNORi treated and untreated as shown in FIGS. 7A-B. Spike S1 protein mice model shows pathological features similar to those observed in autopsy tissue samples from COVID patients (PMID: 3336073).

Cytokine Storm Induced Vascular Dysfunction and Associated Edema are the Hallmark of the COVID-19 Disease.

Under physiological conditions the vascular integrity for selective permeability is maintained by endothelial tight junction proteins for endothelial/endothelial cell interactions (endothelial barrier). Pathological conditions such as inflammatory mediators and hypoxia reduces the levels of tight junction proteins and thus the loss of endothelial (vascular) integrity via decreasing the levels of tight junction proteins. Vascular endothelial barrier integrity is also known to vary in different organs such brain vs other organs depending on the degree of tight junction protein expression. Vascular integrity in brain is known as Blood brain barrier (BBB) due to very selective permeability.

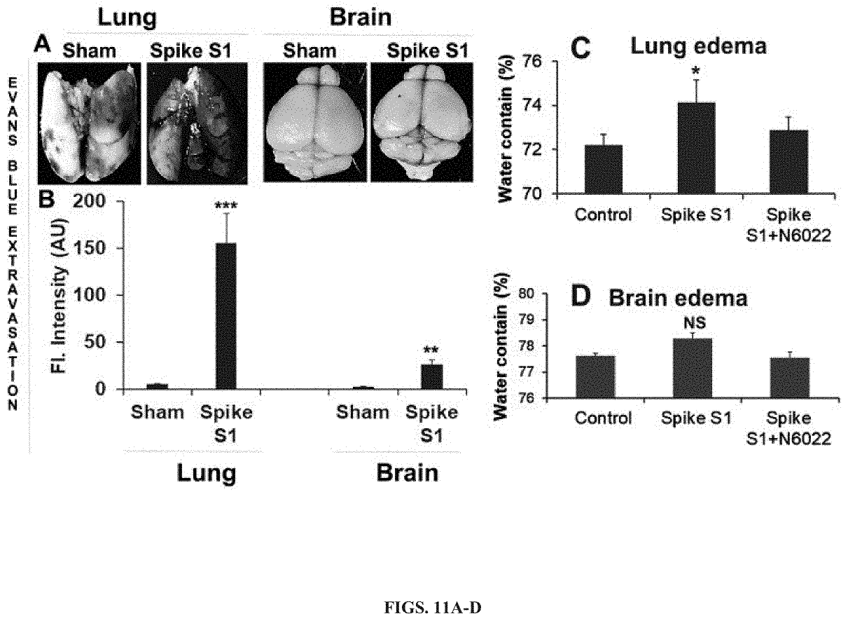

The inventors examined the vascular integrity in spike (S1) protein treated animal as animal model of COVID-19 disease (FIGS. 11A-D) by intravenous injection of EVAN's blue dye. As shown FIG. 11B, there is no extravasation of dye from blood vessels to the lungs as well as the brains of control animals. The blue color on brain and lungs of S1 treated animal represents the extravasation of dye into tissue as a result of disruption of vascular integrity especially in lungs, hall mark of the COVID-19 disease. Therapy with inhibitor of GSNORi (N6022) of S1 treated mice reduced the extravasation blue dye and edema in both lungs as well as the brain by protecting against the S1 protein induced vascular disease pathology. These observations document that GSNORi (N6022) protects against the COVID-19 induced vascular disease pathology and thus potentially the related multiorgan dysfunctions observed in COVID patients.

### Example 3—Discussion

Currently there is no clinically effective therapy for SARS-CoV-2 patients. The supportive treatments including oxygen therapy, conservative fluid management and the use of broad-spectrum antibiotics, for secondary infection, remain the mainstay of therapy. Accordingly, the scientific community is in an accelerated race to develop treatment for SARS-CoV-2 patients.

SARS-COV-2 disease is a multiphasic disease, affecting multiple organ systems, such as 1) viral infection and replication, 2) imbalanced immune response as cytokine storm, and 3) ARDS associated systemic hypoxia and vascular/endothelial dysfunction associated pathologies. Discussed below are potential therapeutic implications of GSNO/GSNORi in SARS-CoV-2 infection and replication, as well as potential efficacy in SARS-CoV-2 associated vascular inflammation, venous and arterial thrombotic complications and systemic hypoxia generated cytokine storm and ARDS.

GSNO and GSNORi and Viral Infection and Viral Replication.

Over the years, the rate of viral infections is associated inflammation. Induction of iNOS and NO is reported to be associated with inhibition of viruses (Coleman J W. Int. Immunopharmacol 1: 1397-1906, 2001; Adler et al., J. Exp. Med. 185: 1533-1540, 1997; Liu et al., BMC Vet. Res. 13:10, 2017). Severe acute respiratory syndrome (SARS) virus is a member of the coronavirus family (SARS CoV-1) and its infection and replication are known to be inhibited by NO compound (SNAP), Organic NO donor (SNAP) and iNOS induced NO were reported to inhibit viral protein and vRNA synthesis and replication of SARS-CoV-1 replication in Vero E6 cells (Akerstrom et al., J. Virol. 79: 1966-1969, 2005). The inventors have previously reported that SNAP and GSNO work via the same mechanism (s-nitrosylation) in animal model of cerebral hypoxia; therefore, GSNO/GSNORi can potentially inhibit SARS-CoV-2 infection and replication.

GSNO/GSNORi for Vascular Inflammation, Venous and Arterial Thromboembolic Complications, Systemic Hypoxia Caused by Cytokine Storm/Respiratory Failure (ARDS) for SARS-CoV-2 Patients.

Respiratory failure induced acute multi-organ failure as a cause of mortality has received relatively little attention for therapeutic intervention in SARS-CoV-2 patients. Infact, ARDS causes disruption of systemic vascular integrity, induce inflammation, venous and arterial thrombosis (pulmonary embolism, deep-vein thrombosis, ischemic stroke, myocardial infarction and/or systemic arterial embolism) leading to multiorgan hypoxia and failure (Klok et al., Thrombosis Res. doi.org/10.1016/j.thromres.2020.04.041).

GSNO was reported to inhibit platelet aggregation, requisite for clot formation (Tsikas et al., FEBS Letts 442: 162-166, 1999; Gordge et al., Br. J. Pharmacol. 124: 141-148, 1998; Crane M S. J. Biol. Chem. 277: 46858-46863, 2002). Moreover, nitrosylating agents like SNAP and GSNO inhibited clotting factor X111, a transglutaminase which catalyzes the cross-linking of fibrin monomers, and in turn inhibited clot formation. This suggests GSNO as one of the regulatory mechanisms for formation of clot with pathophysiological implications (Catani, MV. BBRC 249:275-278, 1998). Further studies using endothelial nitric oxide synthase (eNOS) deletion mutant mice and specific inhibitors of eNOS by Moore and associates (Moore et al., Eur. J. Pharmacol. 651, 152-158) concluded the eNOS produced NO in endothelial cells regulator of platelet function and thus the coagulation cascade, therefore, a potential anti-thrombotic target. Functional endothelium is essential to maintain hemostasis via expression of pro and anticoagulant factors thus underscoring the communication between functional endothelium and control of thrombosis (Yau et al., BMW Cardiovascular Disorders 15: 130-1-11, 2015).

GSNO is known to inhibit vascular/endothelial inflammation as expression of extracellular matrix proteins in cell culture model (Prasad et al., Glia 1:65-77, 2007) and animal model of hypoxia (Khan et al., J. Cereb. Blood Flow Metab. 25:177-192, 2005; Khan et al., J Neurochem. 123:86-97, 2014; Khan et al., J. Neurocem. 2: 86-97, 2012; Khan et al., J Cerebrovasc. Dis. 2019), binding of vascular immune cells to endothelial cells (Prasad et al., Glia 1:65-77, 2007) as well as transmigration of activated immune cells and thus cytokine storm to inflamed tissue/organ (Nath et al., J. Neuroimmune. Pharmacol. 5: 240-251, 2010; Saxena et al., Free Radic. Biol. Med. 121: 57-68, 2018; Choi et al., Nitric Oxide 83: 51-64, 2019 Nittric Oxide 83: 51-64, 2019) and models of traumatic brain and spinal cord injuries (Khan, M and Singh I. Neural. Regen. Res. 6:973-974, 2019; Khan et al., Behav. Brain Res. 340: 63-70, 2018; Choi et al., Nitric Oxide 83: 51-64, 2019 Nitric Oxide 83: 51-64, 2019; Khan et al., Brain Res. 1630: 159-170, 2016). Studies from the inventors' laboratory using cell culture models and animal models of stroke, multiple sclerosis, traumatic brain and spinal cord injuries, we previously reported that GSNO/N6022 inhibit vascular inflammation and protect vascular integrity (Choi. et al., Nitric Oxide 83: 51-64, 2019). These observations provide support for the rationale that exogenous GSNO and endogenous GSNO, generated with use of GSNORi, should optimize GSNO levels that in turn potentially attenuate vascular inflammation and arterial and venous thrombosis observed among SARS-CoV-2 patients.

Hypoxia is known to induce/activate innate immune response and related cell death cellular signaling pathways, just to name a few, in all hypoxic animal model studies. Studies from the inventors' laboratory described that in addition to the above stated pathologies of hypoxia, hypoxia also induces the expression and activity of GSNOR and thus decreases the GSNO levels/homeostasis implying that reduced GSNO levels may be the basis of vascular inflammation and platelet aggregation and clot formation via decreasing the levels of GSNO. They have also reported that GSNO/GSNORi treatments inhibit GSNOR as well as protect against hypoxia pathology via targeting the hypoxic/inflammatory mechanisms in stroke animal model. In summary, based on these data, the inventors believe that GSNO/GSNORi mediated mechanisms should protect against SARS-CoV-2 (ARDS) induced hypoxic and venous and arterial thrombosis pathologies in SARS-CoV-2 patients.

Potential GSNO and GSNORi Therapy for SARS-CoV-2 patients.

As summarized above the GSNO/GSNORi mediated mechanisms target all three phases of SARS-CoV-2 disease: 1) inhibiting viral replication, 2) regulating the immune system by down regulating the proinflammatory mechanisms including inhibition of IL6 while at the same time upregulating the anti-inflammatory mechanisms, and 3) activating GSNO/GSNORi-mediated mechanisms known to protect against hypoxic disease. Therefore, the inventors believe GSNO/GSNORi treatment protects against ARDS associated vascular dysfunctions, systemic hypoxia and multiple organ failure.

Considering that SARS-CoV-2 infection causes multiple mechanistic disease manifestations affecting multiple organs, an effective drug needs to target all critical mechanisms of SARS-CoV-2 disease. Though GDNORi targets only one enzyme—GSNORi—it in turn optimized GSNO levels for S-nitrosylation, targets multiple SARS-CoV-2 disease mechanisms. Targeting the three critical phases of clinical disease of SARS-CoV-2 with GSNO/GSNOi for efficacy provides a more powerful treatment for SARS-CoV-2. So far, GSNO/GSNORi is the only approach that appears to target all three critical phases of the mechanism of SARS-CoV-2 disease.

In summary, the investigations described herein show that GSNO/GSNORi-mediated mechanisms target the three components of the immune (innate, adaptive and autoimmune) system to block “cytokine storm” observed in SARS-CoV-2 disease. The observed dual ability of GSNO/GSNORi to inhibit the production deleterious IL-6 cytokine while upregulating the production of anti-inflammatory beneficial cytokine IL-10. This dual activity of GSNO/GSNORi underscores the ability to these drugs to provide a safe an effective treatment for SARS-CoV-2 patients compared to present day immunosuppressive/immune stimulating drugs.

All of the methods disclosed and claimed herein can be made and executed without undue experimentation in light of the present disclosure. While the compositions and methods of this invention have been described in terms of preferred embodiments, it will be apparent to those of skill in the art that variations may be applied to the methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

1-32. (canceled)

33. A method of treating or preventing viral infection or disease comprising administering an effective amount of S-nitrosoglutathione (GSNO) and/or a GSNO reductase inhibitor to the subject.

34. The method of claim 33, wherein the GSNO reductase inhibitor is N6022, N91115, N6338, Cavonsonstat or SPL-334.1.

35. The method of claim 33, wherein treating results in reduced hypoxia.

36. The method of claim 33, wherein treating upregulates anti-inflammatory innate and adaptive immune response.

37. The method of claim 36, wherein the upregulated anti-inflammatory innate and adaptive immune response comprises IL-10 and/or IL-4.

38. The method of claim 33, wherein treating downregulates pro-inflammatory innate and adaptive immune response.

39. The method of claim 38, wherein the downregulated anti-inflammatory innate and adaptive immune response comprises TNFα, IL-1β and/or IL-6.

40. A method of treating or preventing IL-6 associated disorders in a subject including cancer, a chronic inflammatory disease, an autoimmune disease condition, Castleman's disease, systemic onset juvenile idiopathic arthritis, steroid sparing rheumatoid arthritis, progressive MS, systemic sclerosis, systemic lupus erythematosus, Crohn's disease, neuromyelitis optica spectrum disorders (NMOSD), cytokine release syndrome and multi organ failure comprising administering an effective amount of S-nitrosoglutathione (GSNO) and/or a GSNO reductase inhibitor to the subject.

41. The method of claim 40, wherein the cancer includes IL-6 expressing cancer of breast, prostate, pancreatic, lung, head and neck, colorectal and renal cancers, lymphoma, renal cell carcinoma and hematopoeitic tumors, or a hypoxia associated cancer.

42. The method of claim 33, wherein the one or more symptoms comprise fever, chills, cough, shortness of breath or difficulty breathing, fatigue, muscle or body aches, headache, loss of taste or smell, sore throat, congestion or runny nose, nausea or vomiting, diarrhea, trouble breathing or low blood oxygenation, persistent pain or pressure in the chest, confusion, pneumonia (including viral-induced pneumonia) and acute respiratory distress syndrome (ARDS), blood/vascular, neurological and cognition dysfunctions, or post-COVID-19 disease/dysfunction or vascular and multiorgan COVID-19 disease among “long hauler” COVID patients.

43. The method of claim 33, wherein the subject has an active viral infection.

44. The method of claim 33, wherein the subject at risk of viral infection.

45. The method of claim 33, wherein the subject has tested positive for a viral infection.

46. The method of claim 33, wherein the subject has tested negative for viral infection.

47. The method of claim 33, wherein the GSNO and/or GSNO reductase inhibitor is administered orally, intravenously, intraperitoneally, intratracheally, intratumorally, intramuscularly, endoscopically, percutaneously, subcutaneously, regionally, or by direct injection or perfusion.

48. The method of claim 33, wherein the GSNO and/or GSNO reductase inhibitor is administered orally.

49. The method of claim 33, wherein the subject is a human.

50. The method of claim 33, wherein the subject is a non-human animal.

51. The method of claim 33, wherein said viral infection or disease is a pulmonary viral infection or disease.

52. The method of claim 33, wherein said viral infection or disease is caused by ebolavirus, hantavirus, SARS-CoV-1, SARS-CoV-2/COVID-19, MERS-CoV, influenza virus, Dengue virus, and respiratory syncytial virus.

53. The method of claim 33, wherein the GSNO reductase inhibitor is N6022, N91115, N6338, Cavonsonstat or SPL-334.1.

54. The method of claim 40, wherein the GSNO reductase inhibitor is N6022, N91115, N6338, Cavonsonstat or SPL-334.1
